(19) United States
(12) Patent Application Publication
(10) Pub. No.: US 2025/0269154 A1
(43) Pub. Date: Aug. 28, 2025

(54) INJECTABLE ANGIOPLASTY BALLOON CATHETER COMPRISING UNEQUAL PROXIMAL AND DISTAL BALLOON NECK DIAMETERS

(71) Applicant: Timothy Murphy, Brookline, MA (US)

(72) Inventor: Timothy Murphy, Brookline, MA (US)

(21) Appl. No.: 19/172,724

(22) Filed: Apr. 8, 2025

Related U.S. Application Data
(63) Continuation-in-part of application No. 16/866,907, filed on May 5, 2020, now abandoned.
(63) Continuation-in-part of application No. 18/106,262, filed on Feb. 6, 2023.
(60) Provisional application No. 63/769,055, filed on Mar. 9, 2025.

(51) Int. Cl. A61M 25/10; A61M 25/09

(52) U.S. Cl. CPC A61M 25/104; A61M 25/09; A61M 25/10181; A61M 2025/1063

(57) ABSTRACT

The disclosed invention is in the field of medical devices, namely injectable angioplasty balloon catheters. The embodiments provide an injectable angioplasty balloon catheter that has favorable features of unequal angioplasty balloon proximal and distal neck diameters.

### CROSS-REFERENCE TO RELATED APPLICATIONS

This application claims priority under 35 U.S.C. § 119 and 35 U.S.C. § 120 of provisional patent application No. 63/769,055, confirmation number 4250, filed 9 Mar. 2025, entitled “Injectable Angioplasty Balloon Catheter Comprising Unequal Proximal and Distal Cuff Diameters”, by inventor Timothy Murphy, customer docket number MUR20220203.01, the entirety of which is incorporated herein by reference. This application is a Continuation-In-Part under 37 CFR 1.53(b) of patent applications Ser. No. 16/866,907, EFS ID 39354524, title “Multifunctional Angioplasty and Angiography Catheter Convertible Between Over-the-Wire and Rapid-Exchange Configurations and Methods of Use Thereof”, filed 05-May-2020, is also a Continuation-In-Part of U.S. patent application Ser. No. 18/106,262, EFS ID 47488149, title “Angioplasty Balloon Catheter Comprising A Biaxial-Coaxial Transition,” filed 06-February-2023, all of which are incorporated herein in their entireties, and claims the benefit of the prior applications under 35 U.S.C. § 120.

### FIELD OF THE INVENTION

The present invention relates to medical devices and medical vascular interventional methods. More particularly, the present invention is directed to medical devices used for angioplasty of intravascular stenoses, and methods of use thereof.

### REFERENCES

### U.S. Patents and Patent Applications

Patton U.S. Pat. No. 7,873,404
Patton U.S. Pat. No. 8,532,749
Lentz U.S. Pat. No. 9,011,374
Murphy U.S. Ser. No. 18/106262
Carmel U.S. Pat. No. 10,363,358
Hamilton U.S. Pat. No. 6,129,737
Crocker U.S. Pat. No. 5,470,313
Schulze U.S. Pat. No. 6,056,721

### BACKGROUND

This description of art is not intended to constitute an admission that any patent, publication, or other information referred to is “prior art” with respect to the invention unless specifically designated as such. In addition, this section should not be construed to mean that a search has been made or that no other pertinent information as defined in 37 C.F.R. § 1.56(a) exists.

Angioplasty is a widely used medical procedure for the treatment of vascular occlusions, such as stenotic or narrowed arteries, to restore blood flow. In balloon angioplasty, a catheter that includes an expansile element, viz, an angioplasty balloon, is guided to the target lesion and inflated to compress plaque buildup against the vessel walls, thereby expanding the lumen. Angioplasty balloon catheters comprise an elongated tubular shaft, ranging from 40 cm to 260 cm long, said elongated tubular shaft further comprising a proximal end on which a hub adapter is often affixed and a distal end on which a tapered tip is typically affixed, and said elongated tubular shaft further comprising an expansile member, or angioplasty balloon, said angioplasty balloon typically mounted on said tubular shaft and disposed toward said distal end. Most such angioplasty balloon catheters are designed to be used over a guidewire, and as such typically contain at least two lumens, one for passage of a guidewire therethrough and a second for injection and aspiration of fluid to inflate and deflate said angioplasty balloon. Said angioplasty balloons and catheters are typically made from plastic polymers such as polyethylene, nylon, polytetrafluoroethylene, poly(ether-b-amide), or polyethylene polyamide copolymer, among others. Angioplasty balloons may exhibit compliant, semi-compliant, or noncompliant diameter vs. pressure characteristics.

Said angioplasty balloons may comprise “necks” (also sometimes referred to as “cuffs” or “collars”) which are extensions at their proximal or distal ends that can be used to bond said angioplasty balloons to said tubular shafts. Bonding of components of plastic polymers may be done by thermal means, use of adhesives, or solvent bonding.

Catheter lumen configurations can either be coaxial or multi-axial. A coaxial lumen is where multiple lumens share the same central longitudinal axis and have a configuration of a first inner tube contained within a second outer tube. A multi-axial lumen configuration is where multiple lumens within a tubular element don't share the same central longitudinal axis but rather run with their lumens parallel to each other but not overlapping.

In addition to monofunctional angioplasty balloon catheters, there are multifunctional angioplasty balloon catheters that are injectable and that comprise at least a lumen for a guidewire to pass there through and a lumen for inflation and deflation of an angioplasty balloon, and are able to accommodate injection of fluids through a guidewire lumen even while a guidewire resides within said guidewire lumen throughout its length. Accommodation of injection of fluids has been described in a two lumen catheter by sharing the injection function with a guidewire in an oversized guidewire lumen as in Patton U.S. Pat. No. 8,532,749, or by having a separate third lumen dedicated for injection as in Lentz U.S. Pat. No. 9,011,374. Injection of fluids can be useful during catheter based revascularization procedures because of the need for injection of radiopaque iodine containing solutions to perform angiography, to document blood vessel anatomy and disease. Injection of fluids during revascularization procedures can also be useful to inject medications such as vasodilators, thrombolytics, or anti-neoproliferative medications such as paclitaxel.

### SUMMARY OF THE INVENTION

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

A preferred embodiment of the invention comprises a device and method for an injectable angioplasty balloon catheter that comprises functions of angioplasty balloon inflation and deflation, guidewire passage, and injection of fluids in a single catheter, but with an asymmetric balloon neck diameter design. Such a preferred embodiment of the invention, an injectable angioplasty balloon catheter with unequal neck diameters, comprises an elongated proximal shaft onto which a proximal balloon neck is bonded, said proximal shaft extending at least into an interior of an angioplasty balloon beyond a proximal angioplasty balloon neck bond to said proximal shaft. Said proximal shaft also includes at least a first lumen for passage of a guidewire therethrough, said first lumen also may be shared with fluid injection with a guidewire in place therethrough. In such an exemplary embodiment of a shared guidewire and fluid injection lumen, said first lumen will be oversized by at least 0.003″ diameter on average to allow fluid to be injected around said guidewire while said guidewire is in position throughout a length of said fluid injection lumen between a fluid injection entry port and a fluid injection exit port, and have at least an aperture along a length of said proximal shaft to allow exit of injected fluids to an exterior of said proximal shaft proximal to said angioplasty balloon and to said proximal angioplasty balloon neck bond. Said proximal shaft further contains at least a second lumen for balloon inflation and deflation. Said at least a second balloon inflation and deflation lumen in this exemplary embodiment extends distally beyond said proximal neck bond and to at least one opening or aperture that is in fluid communication with an interior of said angioplasty balloon to allow balloon inflation and deflation.

Distal to said proximal neck bond and distal to said at least an opening or aperture that is in fluid communication with an interior of said angioplasty balloon said proximal shaft terminates in a bond onto a single lumen distal shaft, said distal shaft comprising an inner member within said angioplasty balloon and extending distally to a distal neck bond with said angioplasty balloon distal neck, said distal neck bond may incorporate a bond with a distal tip element. A bond between said proximal shaft and said distal shaft may be a butt joint, lap joint, or other overlapping joint.

Proximal to at least one injection exit port aperture said proximal shaft will be oversized to said guidewire sufficiently to allow fluid injected to pass around said guidewire and distally within said guidewire lumen and exiting said aperture along said length of said proximal shaft. To accomplish that, said guidewire lumen in an exemplary embodiment is oversized compared to the guidewire by an average lumen diameter of at least 0.003″, or between 0.003″ and 0.010″, or up to 0.020″.

In contrast, said distal shaft is sized sufficiently to accommodate a guidewire, but not to accommodate injection or angioplasty balloon inflation and deflation and is therefore of smaller outer diameter than said proximal shaft. An inner diameter of said distal shaft in such an exemplary embodiment may be 0.001″ to 0.003″ greater than said guidewire outer diameter.

The present invention relates to an injectable angioplasty balloon catheter designed to improve the effectiveness of balloon dilation by incorporating an asymmetric configuration in the proximal and distal balloon neck diameters. Specifically, the balloon catheter comprises a proximal neck and a distal neck with different inner and outer diameters, wherein the difference in size between a first proximal neck outer diameter and a second distal neck outer diameter is at least 1 French (0.33 mm) or at least 0.5 French (0.165 mm) or at least 0.3 French (0.1 mm). Said proximal and said distal balloon necks are at least 0.5 mm long, or between 0.5 mm and 100 cm in length prior to bonding to said angioplasty balloon catheter.

In contrast to the design described in U.S. Ser. No. 18/106262, where injection functionality is achieved by having an angioplasty balloon mounted entirely on a distal catheter segment with a smaller outer diameter than a proximal catheter segment, said smaller catheter segment then bonded to said larger proximal catheter segment, said larger proximal segment comprising an oversized guidewire lumen capable of accommodating passage of a guidewire there through while having sufficient mismatch to a guidewire outer diameter to comprise adequate flow of injected fluid to provide satisfactory rate of radiopaque contrast flow to perform diagnostically adequate angiography, in a preferred embodiment of the present invention said angioplasty balloon straddles the bond between proximal shaft and distal shaft, said proximal shaft and said distal shaft having two different outer diameters as described.

Unequal angioplasty balloon cuff diameters can enable a lower profile distal angioplasty balloon bond area compared a proximal bond area, said lower profile distal bond area suitable for entry of said angioplasty balloon into an obstructing lesion in a blood vessel while a larger proximal bond area accommodates a larger proximal catheter segment within the balloon to accommodate the balloon inflation and deflation lumen and fluid injection while over a guidewire in either over-the-wire (OTW) or rapid exchange (RX) configuration.

It will be apparent to those familiar with the art that in order to accommodate injection of fluids along a guidewire within a shared lumen, leaking of fluids from a proximal hub will need to be prevented. That could be accomplished by means of an integral constriction within an integral hub adapter proximal to an injection lumen entry port, or by a modular hub adapter such as a rotating hemostasis valve, Tuohy-borst valve, or similar adapter that enables constriction around a guidewire proximal to an injection entry port.

### BRIEF DESCRIPTION OF THE DRAWINGS

FIG. 1 is a lateral surface rendering of a preferred embodiment of the invention, an angioplasty balloon with unequal balloon neck diameter configuration.

FIG. 2 is a focused cutaway longitudinal view showing an interior of an angioplasty balloon with unequal balloon neck configuration.

FIG. 3 is a surface rendering of an angioplasty balloon with unequal balloon neck diameters not mounted to a catheter.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Devices and related methods are disclosed that generally involve the invention comprising an angioplasty balloon catheter with unequal balloon neck diameter configuration, the means of manufacture of the invention, and the use of the invention during an angioplasty procedure.

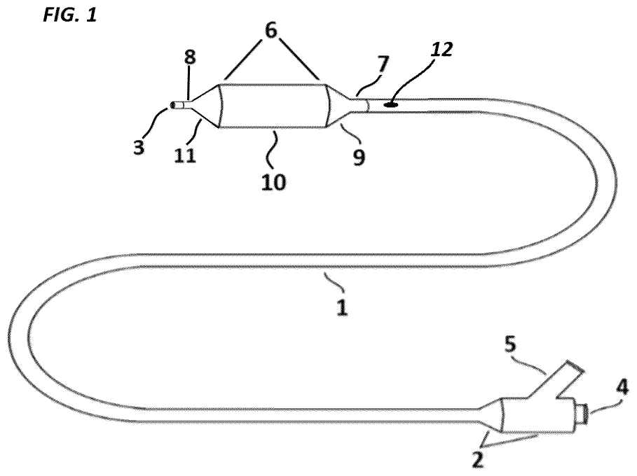

FIG. 1 shows a preferred embodiment of the invention comprising an angioplasty balloon catheter further comprising unequal angioplasty balloon neck configuration, showing a proximal shaft 1, a proximal hub end 2, a distal tip end 3, a proximal guidewire lumen and injection entry port 4, a balloon inflation and deflation port 5, an angioplasty balloon 6, a proximal angioplasty balloon neck 7, a distal angioplasty balloon neck 8, an angioplasty balloon proximal cone 9, an angioplasty balloon middle 10, an angioplasty balloon distal cone 11, and an injection exit port 12. A proximal lumen choke may be present proximal to an injection entry port in some exemplary embodiments (not shown). Said angioplasty balloon proximal neck 7 is larger in inner diameter and in outer diameter than said angioplasty balloon distal neck 8. Image is not drawn to scale, but the intent is to show a proximal neck having a larger diameter than a distal neck by at least 0.3Frence (0.1 mm).

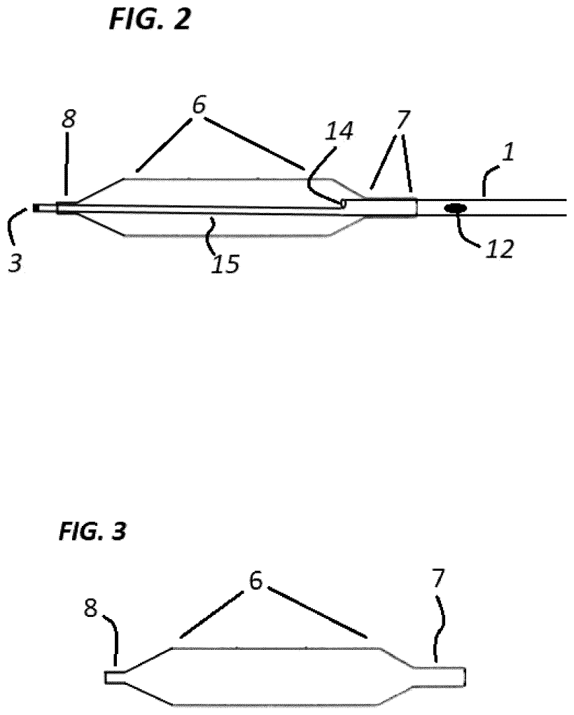

FIG. 2 shows a cutaway longitudinal view of a distal aspect of a preferred embodiment of an angioplasty balloon catheter comprising unequal balloon neck configuration showing an interior of said angioplasty balloon, which illustrates an angioplasty balloon proximal neck 7 with larger inner and outer diameters than an angioplasty balloon distal neck 8. Further illustrated is a proximal shaft 1 that terminates in a balloon inflation lumen port 14, said tubular shaft 1 bonded to a distal shaft 15 within an interior of said angioplasty balloon, said distal shaft 15 being of smaller outer diameter than said proximal shaft, said distal shaft extending to an angioplasty balloon distal neck 8 and bonded thereto, and further extending to said distal tip 3. Said distal shaft 15 may comprise an outer diameter 0.5 French smaller than said proximal shaft 1, or 1.0 French smaller, or 1.5 French smaller. Since said injection exit port 12 is proximal to said angioplasty balloon, said distal shaft 15 does not need to incorporate injection functions or balloon inflation and deflation functions, and is therefore a single lumen dedicated to passage of a guidewire there through, its inner diameter being within five thousands of an inch of its preferred guidewire outer diameter, or within two thousands of an inch or less. In this embodiment said proximal shaft 1 has a multi-axial configuration. Image is not drawn to scale, but the intent is to show a proximal neck 7 having a larger diameter than a distal neck 8 by at least 0.3Frence (0.1 mm).

FIG. 3 is a surface rendering of an exemplary angioplasty balloon not mounted to an angioplasty balloon catheter shaft, illustrating an angioplasty balloon proximal neck 7 with a larger inner and outer diameter than said angioplasty balloon distal neck 8. Image is not drawn to scale, but the intent is to show a proximal cuff having a larger diameter than a distal cuff by at least 0.3Frence (0.1 mm).

### Incorporation by Reference

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

I claim:

1. An angioplasty balloon catheter comprising a tubular shaft disposed between a proximal hub end and a distal tip end, and an expansile member mounted to said tubular shaft disposed toward said distal tip end, said tubular shaft comprising at least two lumens including at least a first lumen for passage of a guidewire or injection of fluids therethrough and at least a second lumen for injection of fluids or gases to inflate and deflate said expansile member, said angioplasty balloon catheter further comprising at least a proximal shaft and at least a distal shaft, said proximal shaft comprising at least one injection exit port proximal to said expansile member, said expansile member further comprising a proximal neck and a distal neck, said proximal neck having a larger outer diameter than said distal neck by at least 0.3 French (0.1 mm).

2. The angioplasty balloon catheter of claim 1 wherein said distal shaft comprises an outer diameter at least 0.5 French smaller than said proximal shaft.

3. The angioplasty balloon catheter of claim 1 wherein said first lumen for passage of a guidewire or injection of fluids therethrough is sized larger than said guidewire between at least an injection entry port and an injection exit port thereby enabling injection of fluids therethrough while a guidewire resides within said first lumen throughout said first lumen's length.

4. The injectable angioplasty balloon catheter of claim 1, wherein said proximal shaft comprises a first outer diameter and said distal shaft comprises a second outer diameter smaller than said proximal shaft first outer diameter by at least 0.3 French (0.1 mm).

5. The injectable angioplasty balloon catheter of claim 1, wherein said proximal neck is at least 0.5 mm long.

6. The injectable angioplasty balloon catheter of claim 1, wherein said distal neck is at least 0.5 mm long.

7. A method of performing an angioplasty in a patient comprising:
obtaining guidewire access into a vascular system and across an obstructing lesion to be treated;
introducing an injectable angioplasty balloon catheter comprising an expansile member disposed toward a distal tip end, said expansile member further comprising a proximal neck and a distal neck, said proximal neck having a larger inner diameter than said distal neck by at least 0.3 French (0.1 mm), into the vascular system;
advancing said injectable angioplasty balloon catheter such that an angioplasty balloon is located within an obstructing lesion;
inflating said angioplasty balloon within said obstructing lesion;
deflating said angioplasty balloon;
removing said injectable angioplasty balloon catheter from said patient's body.
